(19) United States
(12) Patent Application Publication
(10) Pub. No.: US 2005/0034175 A1
(43) Pub. Date: Feb. 10, 2005

(54) Immune cloning and uses thereof

(76) Inventors: Zuhair A. Latif, Vernon Hills, IL (US); Sean F. Nowlan, Winthrop Harbor, IL (US)

Correspondence Address: ROBERT DEBERARDINE;ABBOTT LABORATORIES, 100 ABBOTT PARK ROAD, DEPT. 377/AP6A, ABBOTT PARK, IL, 60064-6008, US

(21) Appl. No.: 10/654,724

(22) Filed: Sep. 4, 2003

Related U.S. Application Data
(60) Provisional application No. 60/409,327, filed on Sep. 9, 2002.

(51) Int. Cl. A01K 67/027

(52) U.S. Cl. 800/6; 800/14; 800/15; 800/16; 800/17; 800/18

(57) ABSTRACT

The subject invention relates to a method of creating a cloned animal having the precise immunological response and capabilities as the founder animal.

The present application claims priority to U.S. Provisional Application No. 60/409,327, filed on Sep. 2, 2002, hereby incorporated in its entirety by reference.

### BACKGROUND OF THE INVENTION

### Technical Field

The subject invention relates to a method of cloning an immune response. In particular, such a method allows one to produce a cloned mammal with a predicted immunological profile.

### BACKGROUND INFORMATION

Normally, cells in an animal undergo mutations. Some of these mutations result in malignancy, while others are silent and cause no adverse effect. In some cells, silent mutations are either difficult to identify or are entirely undetectable. In other cells, however, such mutations are detected and measured by the type and amount of a by-product of that mutation (e.g., immunoglobulins).

Due to differences in growth characteristics and function, some cells are more prone to mutations than others. For example, fibroblasts are known to have very low mutation rates, while lymphocytes, which are designed to protect the organism from external (and internal) invaders, by necessity have higher mutation rates. Lymphocytes mount immune responses by adapting their genes to better protect against new invaders. Such adaptations are perfected only after exposure to antigen. Thus, the immune response is a learning or adaptive process affected by somatic mutations in a select subpopulation of lymphocytes. Once these undifferentiated but antigen-selected cells differentiate into mature antibody-producing cells (e.g., plasma and memory cells), they are committed to making only those specific molecules for the remainder of their lives and, as such, their genes are rearranged irreversibly and permanently. Thus, when a specific immune response is needed, using these committed cells to clone the immune response results in production of an “immune clone”.

Animal cloning is achieved by transferring the nucleus from the cell of a founder animal to an enucleated ovum of a surrogate mother, and then implanting the newly creating hybrid cell into the uterus of a surrogate mother during estrous. (See Cloning, Vol. 1, Number 3, pp. 161-170, 1999; see also K. Hochedlinger et al., Nature 718:1-4(2002)). Since animal cloning is based on the need or desire to asexually reproduce an animal with an identical set of genes as the founder animal, it is necessary to select a cell type from the founder animal that is least likely to have undergone mutations. This will ensure that the original set of genes that produced the founder animal is replicated to produce a clone or an “identical” animal as the founder. The cell type that has been most successfully used for this purpose is the fibroblast.

Certainly there exists a need to create a cloned animal having the same immune response and immunological characteristics as the founder animal. Thus, a proven immune response in the founder will then be reproduced in the clone. Only by such a method can one ensure that the antibodies or cells produced by the founder and having a particular specificity, affinity, etc. will, in turn, be produced by the clone. Such a method is provided by the present invention.

### SUMMARY OF THE INVENTION

The present invention includes a method of creating an immune response in a clone that is identical to the immune response of a founder mammal. This method comprises the steps of immunizing a founder mammal with an immunogen or antigen; isolating a lymphocyte from the immunized founder mammal and utilizing the isolated lymphocyte as a source of nuclear material to construct a clone; and allowing the constructed clone to develop to maturity such that the immune response of the clone is identical to the immune response of the founder mammal. In particular, the lymphocyte of the founder mammal is transferred to an enucleated ovum of a surrogate female, and the resulting embryo is transferred into the uterus of a surrogate female during estrous. The founder and cloned mammal or animal may be, for example, a mouse, a rabbit, a sheep, a horse, a pig or a cow. The immune response of the clone results in production of antibodies upon exposure to the immunogen. The lymphocyte may be, for example, a peripheral blood lymphocyte, a lymph node lymphocyte, a splenocyte or a bone marrow cell. The immunogen may be, for example, an antigen, an epitope, a hapten, or an immunogenic portion of any one of these entities. For purposes of the present invention, a “portion” refers to a part of the immunogen which is able to elicit the same desirable immunological response (for example, production of antibodies) as the full immunogen.

### BRIEF DESCRIPTION OF THE FIGURES

FIG. 1 illustrates the method of the present invention in which the founder animal is immunized, cells are isolated and purified from the founder animal, the nucleus of a lymphocyte from the founder animal is transferred to an enucleated oocyte of a surrogate animal, and the resulting blastocyst is transferred into the uterus of the surrogate.

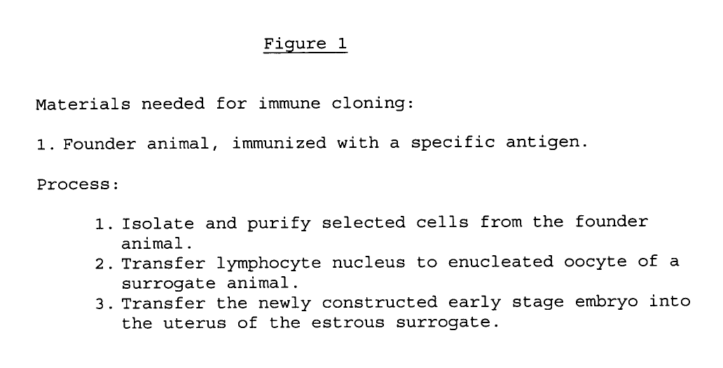

### DETAILED DESCRIPTION OF THE INVENTION

For purposes of the present invention, the need for immune cloning is not to reproduce the animal itself, but rather to produce a new animal with the same immune capacity and immunological identity as the founder animal. Where a particular immune response is rare or difficult to replicate, it is advantageous to use immune lymphocytes as the source of nuclear transfer, in accordance with the present invention. More specifically, when the desired and correct lymphocyte is selected from the founder animal and then used for cloning, the process will result in an “immune clone” with the identical ability to mount the same, unique immune response as the founder.

Generally, the method of the present invention is carried out as follows:
1) The founder animal is immunized with an antigen of interest (e.g., a hapten, epitope, immunogenic portion of an antigen, cell receptor, etc.).
2) Lymphocytes produced as a result of this immunization are sorted in order to isolate B or T cells specific for the antigen.
3) The nucleus of an isolated B or T lymphocyte (of step (2)) is transferred to an enucleated oocyte of an estrous surrogate female of the same species.
4) The embryo (early blastocyst) is transferred into the uterus of the estrous surrogate. Implantation occurs as a result of the transfer.
5) The embryo is allowed to gestate, and the surrogate gives birth to the fetus (i.e., immune clone) after proper development thereof.

Once the immune clone has reached adulthood, it is challenged with the initial antigen used to immunize the founder animal. One then compares the antibody specificity and titer of the immune clone (produced in connection with this specific antigen) with the antibody specificity and titer of a challenged fibroblast clone (produced using a fibroblast cell from an immunized founder animal as the nuclear donor). If immune cloning is successful, the immune clone will show a secondary (or amamnestic) immune response, while the fibroblast clone will show only a primary immune response. More specifically, primary immune responses are slow to develop, usually result in low titer and low specificity antibody. On the other hand, a secondary immune response is faster to develop, is characterized by high antibody titer and high specificity. This is the main difference between the two responses and is the basis for the common vaccination. Thus, the immune response of the founder has been cloned, and the immune system of the clone should be virtually identical, in terms of function and response, to that of the founder animal. For purposes of the present invention, a “founder animal” is one that is known, following experimentation, to produce a unique immune response that is difficult to duplicate in other animals of the same or different species.

The need for the present invention is significant. Such a need may be, for example, illustrated as follows: An essential and critical component of a diagnostic assay for T4 is sheep anti-T4 serum that is immobilized onto a solid phase (e.g., microparticles, microtiter wells, beads, etc.). In combination with a conjugate made up of T3 (Triiodothyronine, an analog of T4) and alkaline phosphatase, the sheep serum confers basic, critical, quality attributes required to generate a distinct standard calibration curve and allows for an estimate of Free Thyroxin 4(FT4) in patient samples.

The serum is developed by immunizing sheep with T4-Tg complex. Thyroxin (T4) is coupled onto a protein carrier molecule (e.g., porcine thryoglobulin or Tg), then emulsified in an adjuvant (e.g., aluminum hydroxide, N-acteyl-muramyl-L-threonyl-D-isoglutamine or N-acetyl-nornuramyl-L-alanyl-D-isoglutamine) prior to injection into sheep. This is a classical approach to raising needed immune responses in experimental animals. Historically, however, this method of immunization produced antibodies recognizing T4molecules; yet, in the great majority of instances, the resulting antibodies did not perform adequately in diagnostic tests.

By creating an immune clone, one may produce antibodies having the same properties as those produced by the founder animal (i.e., having the ability to recognize T4molecules and which perform properly in diagnostic assays or for other uses as well (e.g., vaccines, immunotherapy, etc.)).

The method of creating an immune clone is particularly useful to generate and propagate a specific immune response (e.g., antibody or cell-mediated) in an animal when the response is difficult to reproduce (e.g., when one wishes to generate antibodies to a very small antigen or one with an unusual tertiary structure).

Further, using the immune clone, one may produce rare reagents (e.g., anti-viral antibodies, anti-bacterial antibodies and anti-fungal antibodies). Basically, the antigen of choice is utilized in creating the founder animal, which is then used as a source of lymphocyte, which is, in turn, used to as a source of nuclear material for creation of the immune clone. Lymphocytes may be enriched or specifically isolated (using panning or sorting techniques, for example) to ensure a higher likelihood of using the correct cell with the unique immune characteristics. In view of the above, immune cloning not only produces an animal with a specific immune potential but also obviates the need for primary immunization.

In terms of the antigen used for immunization of the founder animal and subsequent challenge of the Immune Clone, 30 one may use any immunogen or a portion thereof capable of eliciting an immune response. If a portion of an immunogen is used, it must be able to elicit the desirable immune response of the full immunogen. Examples of suitable antigens or immunogens include haptens, epitopes, anti-antibodies, cell receptors, etc. For purposes of immunization and challenge, the antigen may be mixed with a pharmaceutically acceptable excipient such as, for example, oil, water, saline, dextrose, glycerol, ethanol, or mixtures thereof. The vaccine may also contain small amounts of additional substances such as wetting or emulsifying reagents, pH buffering agents and/or adjuvants which enhance the effectiveness of the vaccine. Examples of suitable adjuvants include, for instance, AlOH, N-acetyl-muramyl-L-threonlyl-D-isoglutamine (thr-DMP) and N-acetyl-nornuramyl-L-alanyl-D-isoglutamine.

Further, the method of the present invention may be used in connection with mammals of many species including, for example, horses, sheep, cows, pigs, rabbits, mice, etc. Either domestic or non-domestic animals may be used.

Additionally, based upon the method of the present invention, one may produce several immune clones having the same immunological response as the founder animal. Consequently, one may produce an endless supply of antibodies (or cells produced in response to an immunogen) without the concern of initiating the desired immunological response in other animals. Without such an immune clone, one could not guarantee the supply of the founder animal's antibodies as the founder animal could become ill or die. Such an event would obviously prevent the use of the appropriate type of antibody in antigen assays, passive vaccines, development of therapeutics, etc. or in any situation in which a consistent and specific immune response must be duplicated.

The present invention may be illustrated by the use of the following non-limiting example:

### EXAMPLE I

### Cloning from an Immune B Lymphocyte (I.E., Immune Cloning)

Initially, fucosyltransferase transgenic (FTT) (or other transgenic) mice are immunized with an antigen such as T4-TG. B-lymphocytes are then selected which produce antibodies specific for the T4 hapten, by panning, sorting (with FITC-T4stained lymphocytes) or other techniques known to those of ordinary skill in the art. Selected lymphocytes (i.e., immune B cells) are then used as nuclear donors to develop cloned mice (i.e., the immune clones). At adulthood, the immune clone is challenged with T4-TG. Antibody titer to T4 is measured. A fibroblast clone (i.e., a cloned mouse created using a fibroblast cell obtained from immunized mice as a nuclear donor) is also challenged with T4-TG, and antibody titer to T4 is measured. The two responses are compared. If immune cloning is successful, the immune clone will show a secondary immune response, while the fibroblast clone will show only a primary immune response.

1. A method of creating an immune response in a clone that is identical to the immune response of a founder mammal comprising the steps of:
a) immunizing a founder mammal with an immunogen;
b) isolating a lymphocyte from said immunized founder mammal and utilizing said isolated lymphocyte as a source of nuclear material to construct a clone; and
c) allowing said constructed clone to develop to maturity such that said immune response of said clone is identical to said immune response of said founder mammal.

2. The method of claim 1 wherein said mammal is selected from the group consisting of a mouse, a rabbit, a sheep, a horse, a pig and a cow.

3. The method of claim 1 wherein said immune response of said clone results in production of antibodies upon exposure to said immunogen.

4. The method of claim 1 wherein said lymphocyte is selected from the group consisting of a peripheral blood lymphocyte, a lymph node lymphocyte, a splenocyte and a bone marrow cell.

5. The method of claim 4 wherein said lymphocyte is a B lymphocyte.

6. The method of claim 1 wherein said immunogen is selected from the group consisting of an antigen, an epitope, a hapten, and an immunogenic portion thereof.

7. The method of claim 1 wherein said lymphocyte of step (b) of said founder mammal is transferred to an enucleated ovum of a surrogate female, and the resulting embryo is transferred into the uterus of a surrogate female during estrous.
